(19) United States
(12) Patent Application Publication
(10) Pub. No.: US 2001/0039265 A1
(43) Pub. Date: Nov. 8, 2001

(54) Autism

(76) Inventor: Stephen Dealler, Burnley (GB)

Correspondence Address: BEYER WEAVER & THOMAS LLP, P.O. BOX 778, BERKELEY, CA, 94704-0778, US

(21) Appl. No.: 09/775,933

(22) Filed: Feb. 2, 2001

(30) Foreign Application Priority Data
Feb. 11, 2000 (GB) 0003048.6

(51) Int. Cl. A61K 31/727; A61K 31/726; A61K 31/737

(52) U.S. Cl. 514/54; 514/56

(57) ABSTRACT

The present invention relates to a method for combatting the symptoms of autism in a subject, said method comprising: administering to the subject a polyanionic polyglycoside (eg a polysulphonated polyglycoside).

The present invention relates to a method for combatting autism using polyanionic polyglycosides (eg polysulphonated polyglycosides).

Autism is a childhood psychosis originating in infancy which is characterised by a wide spectrum of psychological symptoms that progress with age (eg lack of responsiveness in social relationships, language abnormality and a need for constant environmental input). It generally appears in children between the ages of two and three years and gives rise to a loss of the development previously gained by the child. The syndrome frequently leads to repeated narrow spectrum diets and psychological difficulties in changing other aspects of life. Epilepsy commonly develops after the age of ten and many drugs are used to control this. The child may develop into an adult that can not be involved normally in society or generate its own income.

Autism is commonly associated with certain abdominal complaints (such as abdominal pain, nausea, retching, constipation, inflammatory bowel disease and malabsorption). The abdominal complaints have been looked upon for some time as being separate to the psychological symptoms or caused by them (Wing, 1997, Autism, 1, 13 to 23) and have therefore undergone little investigation.

The recent increase in the number of cases of autism reported in the UK and the USA has suggested that some factor apart from a psychological one is involved in its pathogenesis. The presence of peptides with opioid properties in the diet and urine of autistic subjects are consistent with the hypothesis that the psychological syndrome could be the result of the uptake of neuroactive compounds from the gut (Reichelt et al, 1991, Brain Dysfunct., 4, 308-319 and Reichelt et al, 1994, Dev. Brain Dysfunct., 7, 71-85). The finding that the gut absorbs compounds that are not absorbed in matched controls has suggested that in autistic subjects, diet-derived opioids may reach the brain as a result of increased permeability of the gut (ie a “leaky gut”) in which the barrier between the gut lumen and the blood is inadequate. It has been reported that the psychological symptoms can be modified by the diet but the precise diet that is needed has been difficult to maintain and the patient commonly regresses when the diet stops (Reichelt et al, 1991, Brain Dysfunct., 4, 308-319).

Although it might have been expected that therapeutic agents which alter the uptake of neuroactive compounds from the gut would alter the psychological symptoms of the autistic condition, the effects of such therapeutic agents have in fact been unpredictable. For example, for the hormone secretin (which is known to have an effect on the growth and development of the gut and to alter the ability of the gut to absorb and prevent absorption of certain compounds), a positive effect on autism has been seen in some children but inexplicably not in others. Other treatments for autism have been mainly psychological since neurologically active therapeutic agents have not been shown to have great effect.

The reported finding of excess inflammation in the colon, oesophagus and duodenum of patients with autism (Wakefield et al, 1998, Lancet, 351, 637 to 641 and Wakefield et al, 1998, Lancet, 351, 351 to 352) when no standard cause was found and none found in controls has yet to be explained. In addition, findings of low level activity of sulphotransferase (enzymes which donate sulphate anions to certain macromolecules such as mucins, glycoproteins and glycosaminoglycans) in autistic children and yet extremely low levels of inorganic sulphate ions in plasma have also not been explained (see Alberti et al, 1999, Biol. Psychiatry, 46, 420-424 and Waring et al, 1997, Dev Brain Dysfunct, 10, 40-43).

Consistent with these observation, the present invention is based on the recognition that certain polyglycosides which inhibit inflammation of the gut lead to an improvement in the psychological symptoms of an autistic subject. More particularly, such polyglycosides are thought to interact with heparan binding sites to prevent or inhibit inflammation of the gut so as to improve the psychological symptoms of an autistic subject. The beneficial effect of such compounds is thought to be closely associated with the involvement of the sulphate metabolism in the pathogenesis of the gut abnormality (eg the sulphation of macromolecules such as glysoaminoglycans).

Thus viewed from one aspect the present invention provides a method for combatting the symptoms of autism in a subject, said method comprising:

administering to the subject an effective amount of a polyglycoside or precursor thereof (or a mixture of polyglycosides or a mixture of precursors of polyglycosides) or a salt thereof (eg a calcium, sodium, magnesium, potassium or ammonium salt thereof), wherein the polyglycoside comprises a chain of glycosidic residues substituted regularly or irregularly with an anionic substituent.

In a preferred embodiment, the polyglycoside is capable of interacting with a cellular heparan binding site.

In a preferred embodiment, the polyglycoside is capable of entering the intercellular fluid.

In a preferred embodiment, the polyglycoside is capable of restoring disrupted tight junctions between epithelial cells.

In a preferred embodiment, the chain of glycosidic residues comprises a glucosamine residue.

In a preferred embodiment, the chain of glycosidic residues comprises a iduronic acid residue.

In a preferred embodiment, the chain of glycosidic residues comprises a glucoronic acid residue.

In a particularly preferred embodiment, the chain of glycosidic residues comprises a glucosamine residue and either a glucoronic acid or an iduronic acid residue.

In a preferred embodiment, the chain of glycosidic residues comprises a xylose residue.

Where the glycosidic chain is substituted regularly, it may comprise a repeating glycosidic unit. Preferably the repeating glycosidic unit is a mono-, di-, tri- or polysaccharide unit.

The anionic substituent may be selected from the group consisting of sulphate (eg SO₃⁻ or HSO₃) and carboxylate (eg CO₂⁻ or HCO₂). In a preferred embodiment, the anionic substituent is sulphate. In this case, there may be one, two or three sulphate substituents per glycosidic residue.

The anionic substituent may be directly or indirectly ring-bound. For example, the anionic substituent may be bound to the ring by a bridging group such as for example an epoxy group, an optionally alkyl-substituted imino group or an alkoxo (eg —CH₂O—) group.

The polyglycoside may be a natural or synthetic polyglycoside and may be homogeneous or heterogeneous. The glycosidic chain may be linear or non-linear. Typically the molecular weight of the polyglycoside is in the range 1 kDa to 8 MDa.

Macromolecules are able to pass from the gut lumen into the blood by traversing epithelial cells but cannot normally pass between cells due to the presence of tight junctions. Toxins and inflammation are two potential causes of disruption of tight junctions. Once disrupted, the effect will tend to be potentiated by the enhanced uptake of further toxins and/or the inflammatory response resulting from the initial breach of the epithelial barrier. Without wishing to be bound by any theoretical considerations, it is thought that preventing or reducing inflammation in accordance with the invention leads to an improvement of the tight junctions found between gut epithelial cells which thereby inhibits the uptake of neouroactive compounds that may be causing the psychological symptoms of autism. The polyanionic polyglycosides are thought to interact with cellular heparan binding sites which are found on proteins on the cell surface. As these proteins are taken into the cell to be either destroyed or recycled, any compound bound to the heparan binding site enters the cell and has an effect. For instance, specific cytotoxic cytokinins may cause apoptosis in epithelial cells and lead to severe ulceration and further inflammation of the gut surface. Polyanionic polyglycosides present in the intercellular fluid also bind with heparan binding sites (and some would displace the normal compounds such as heparin) on cytokinins and when they themselves are taken into the cells, they are destroyed with the cytokinin and no effect by these compounds is seen. As such, the presence of polyanionic polyglycosides will prevent inflammation and damage caused by cellular chemicals.

Specific examples of polyglycosides include heparin and salts thereof (eg calcium, sodium, magnesium or potassium salts thereof, preferably calcium and sodium salts thereof), low molecular weight fragments of heparin (obtainable by chemical or enzymatic depolymerisation of heparin) and salts thereof (eg sodium and calcium salts thereof) and heparinoids and salts thereof. By heparinoids is meant heparin derivatives including naturally occurring or synthetic highly sulphated polysaccharides of similar structure. These include sulphated glucosaminoglycans, glycosaminoglycan polysulphates and sulphated mucopolysaccharides; heparan sulphate, dermatan sulphate, chondroitin 4-sulphate and chondroitin 6-sulphate (and salts (eg sodium salts) and mixtures thereof); pentosan polysulphate sodium, sodium apolate and sulodexide.

Further specific examples of polyglycosides include λ-carrageenan, κ-carrageenan and τ-carrageenan (and mixtures thereof), dextran sulphate and salts thereof (eg sodium and potassium salts thereof, preferably sodium salts thereof); sulphated polyhyaluronic acid; colominic acid sulphate; and taurine.

Mixtures of these polyglycosides may also be used in accordance with the invention.

Preferred polyglycosides for use in accordance with the invention are carrageenans, dextran sulphate and salts thereof which are thought to beneficially displace heparin and heparan from binding with binding sites on inflammatory molecules. Particularly preferred is pentosan polysulphate sodium which beneficially displaces heparin from binding sites and is advantageously orally administrable and is absorbed to a low degree. Pentosan polysulphate sodium is available (for example) in capsule form commercially under the trade mark Elmiron (Norton Healthcare).

The polyglycoside may be formulated as desired with conventional buffers, emulsifiers, stabilisers, viscosity enhancers, inert ingredients (such as excipients), additives and flavourings.

The polyglycoside may be administered by any conventional route such as parenterally or enterally including orally, rectally or intraveneously. The preferred administration route for the chosen polyglycoside may be readily determined by the skilled person and oral administration is generally preferred.

The polyglycoside may be administered in any convenient form such as capsule or tablet (preferably enterically coated) or syrup. The actual administration form for the chosen polyglycoside may be readily determined by the skilled person.

In a first preferred embodiment, the polyglycoside is administered in a novel sustained release formulation (eg in capsule or tablet form), particularly preferably a sustained release formulation which is adapted to release the polyglycoside in the ileum or colon (thereby reducing the number of daily doses).

A sustained release formulation which releases the active ingredient over a period of time (preferably in the ileum or colon) and thereby reduces the number of daily doses is advantageous. Such a sustained release formulation may use any conventional sustained release component such as (for example) a barrier, coating or erodable matrix.

In a second preferred embodiment, the polyglycoside is administered in a novel enterically coated unit (eg a unit in the form of an enterically coated capsule or tablet or in the form of enterically coated beads, pellets or granules contained in (for example) a tablet or capsule).

Viewed from a further aspect the present invention provides an orally administrable, sustained release formulation comprising a polyglycoside (or a mixture of polyglycosides) or a salt thereof (eg a calcium, sodium, magnesium, potassium or ammonium salt thereof) as hereinbefore defined.

Viewed from a yet further aspect the present invention provides an orally administrable formulation comprising a polyglycoside (or a mixture of polyglycosides) or a salt thereof (eg a calcium, sodium, magnesium, potassium or ammonium salt thereof) as hereinbefore defined in the form of an enterically coated unit (eg a unit in the form of an enterically coated capsule or tablet or in the form of enterically coated beads, pellets or granules contained in (for example) a tablet or capsule).

The polyglycoside may be administered in a dose which depends on the weight of the subject. This is readily determined by a person skilled in the art for the chosen polyglycoside. Typically the daily dose for a child under three years would be 50 mg orally and for a child between three and five years would be 100 mg orally. For a subject from the age of 6 years upwards the dose would typically be 100 mg twice daily. If diarrhoea results, the dose should be reduced.

Typically the selected dose is administered three times daily (eg orally) and the blood should be monitored to ensure that large amounts are not being absorbed after 48 hours. The administration regime will typically continue for several months (eg three to six months or longer) before any long term benefit is observed.

Viewed from a still further aspect the present invention provides an orally administrable formulation comprising a polyglycoside (or a mixture of polyglycosides) or a salt thereof (eg a calcium, sodium, magnesium, potassium or ammonium salt thereof) as hereinbefore defined, together with one or more carriers or excipients, wherein the polyglycoside is capable of entering the intercellular fluid and is present in an amount sufficient to therapeutically inhibit the psychological symptoms of autism.

1. A method for combatting the symptoms of autism in a subject, said method comprising:
administering to the subject an effective amount of a polyglycoside (or a mixture of polyglycosides) or a salt thereof, wherein the polyglycoside comprises a chain of glycosidic residues substituted regularly or irregularly with an anionic substituent.

2. The method as claimed in claim 1 wherein the salt is a calcium, sodium, magnesium, potassium or ammonium salt.

3. The method as claimed in claim 1 wherein the polyglycoside is capable of interacting with a cellular heparan binding site.

4. The method as claimed in claim 1 wherein the chain of glycosidic residues comprises a glucosamine residue.

5. The method as claimed in claim 1 wherein the chain of glycosidic residues comprises an iduronic acid residue.

6. The method as claimed in claim 1 wherein the chain of glycosidic residues comprises a glucoronic acid residue.

7. The method as claimed in claim 1 wherein the chain of glycosidic residues comprises a glucosamine residue and either a glucoronic acid or an iduronic acid residue.

8. The method as claimed in claim 1 wherein the chain of glycosidic residues comprises a xylose residue.

9. The method as claimed in claim 1 wherein the glycosidic chain is substituted substantially regularly and comprises a repeating glycosidic unit being a mono-, di-, tri- or polysaccharide unit.

10. The method as claimed in claim 1 wherein the anionic substituent is selected from the group consisting of sulphate and carboxylate.

11. The method as claimed in claim 10 wherein the anionic substituent is sulphate.

12. The method as claimed in claim 11 wherein the glycosidic chain comprises one, two or three sulphate substituents per glycosidic residue.

13. The method as claimed in claim 1 wherein the anionic substituent is bound to the ring by a bridging group.

14. The method as claimed in claim 13 wherein the bridging group is an epoxy group, an optionally alkyl-substituted imino group or an alkoxo group.

15. The method as claimed in claim 14 wherein the bridging group is an epoxy group.

16. The method as claimed in claim 1 wherein the polyglycoside is selected from the group consisting of heparin and salts thereof, low molecular weight fragments of heparin and salts thereof and heparinoids and salts thereof.

17. The method as claimed in claim 16 wherein the heparinoid is selected from the group consisting of sulphated glucosaminoglycans, glycosaminoglycan polysulphates, sulphated mucopolysaccharides, heparan sulphate, dermatan sulphate, chondroitin 4-sulphate, chondroitin 6-sulphate, pentosan polysulphate sodium, sodium apolate and sulodexide.

18. The method as claimed in claim 1 wherein the polyglycoside is selected from the group consisting of λ-carrageenan, κ-carrageenan, τ-carrageenan (and mixtures thereof), dextran sulphate and salts thereof, sulphated polyhyaluronic acid, colominic acid sulphate and taurine.

19. The method as claimed in claim 16 wherein the salt is a calcium or sodium salt.

20. The method as claimed in claim 18 wherein the polyglycoside is selected from the group consisting of λ-carrageenan, κ-carrageenan, τ-carrageenan and dextran sulphate and salts thereof.

21. The method as claimed in claim 16 wherein the polyglycoside is pentosan polysulphate sodium.

22. The method as claimed in claim 1 wherein the polyglycoside is in an orally administrable formulation.

23. The method as claimed in claim 22 wherein the orally administrable formulation is a sustained release formulation.

24. The method as claimed in claim 23 wherein the sustained release formulation is adapted to release the polyglycoside in the ileum or colon.

25. The method as claimed in claim 22 wherein the orally administrable formulation is in an enterically coated unit.

26. The method as claimed in claim 25 wherein the enterically coated unit is in the form of an enterically coated capsule or tablet or in the form of enterically coated beads, pellets or granules contained in a tablet or capsule.

27. An orally administrable, sustained release formulation comprising a polyglycoside (or a mixture of polyglycosides) or a salt thereof, wherein the polyglycoside comprises a chain of glycosidic residues substituted regularly or irregularly with an anionic substituent.

28. An orally administrable formulation comprising a polyglycoside (or a mixture of polyglycosides) or a salt thereof, wherein the polyglycoside comprises a chain of glycosidic residues substituted regularly or irregularly with an anionic substituent, said formulation being in the form of an enterically coated unit.

29. An orally administrable formulation as claimed in claim 28 wherein the enterically coated unit is an enterically coated capsule or tablet or enterically coated beads, pellets or granules contained in a tablet or capsule.

30. An orally administrable formulation comprising a polyglycoside (or a mixture of polyglycosides) or a salt thereof, together with one or more carriers or excipients, wherein the polyglycoside comprises a chain of glycosidic residues substituted regularly or irregularly with an anionic substituent and is capable of entering the intercellular fluid, said polyglycoside being present in an amount sufficient to therapeutically inhibit the psychological symptoms of autism.
